(19) United States
(12) Patent Application Publication
(10) Pub. No.: US 2025/0135099 A1
(43) Pub. Date: May 1, 2025

(54) Infusion Consumable, Infusion Device, and Method for Detecting the Model of the Infusion Consumable

(71) Applicant: Medcaptain Medical Technology Co., Ltd., Shenzhen (CN)

(72) Inventors: Xiang LI, Shenzhen (CN); Pan HU, Shenzhen (CN); Jun DONG, Shenzhen (CN)

(73) Assignee: Medcaptain Medical Technology Co., Ltd., Shenzhen (CN)

(21) Appl. No.: 18/928,025

(22) Filed: Oct. 26, 2024

(30) Foreign Application Priority Data
Oct. 26, 2023 (CN) 202311404885.7

(51) Int. Cl. A61M 5/14; A61M 5/168

(52) U.S. Cl. CPC A61M 5/1415; A61M 5/16831

(57) ABSTRACT

An infusion consumable for use in an infusion device includes a mounting portion, which is provided with a mounting hole. The mounting hole is provided with an identification member, and a depth of the mounting hole is greater than a thickness of the identification member. The type of consumable is detected based on characteristics, such as insertion depth and/or magnetic properties, relative to the mounting hole.

### REFERENCE TO RELATED APPLICATIONS

The invention claims priority of Chinese Patent Application No. 202311404885.7, entitled “Infusion Consumable, Infusion Device, and Method for Detecting Model of Infusion Consumable” filed with the China National Intellectual Property Administration on Oct. 26, 2023, which is incorporated herein by reference in its entirety.

### TECHNICAL FIELD

This invention generally relates to medical devices, and more particularly, to an infusion consumable, an infusion device, and a method for detecting a model of the infusion consumable.

### BACKGROUND ART

A fluid can be fed to the patient via a nutrient pump to meet the patient's needs. For example, the fluid may be a nutrient solution.

Typically, a nutrient pump can accommodate various models of infusion consumables. Once the infusion consumable is mounted on the pump, the nutrient pump can automatically identify the specific model of the consumable, which allows it to determine the infusion mode according to the consumable model. In the prior art, infusion consumables are divided into two main categories. The first category includes two types of consumables: those with an identification component and those without. A nutrient pump that matches the first category of consumables generally only needs one sensor to detect the identification component. The second category includes multiple models of infusion consumables, each equipped with a different type of identification component. A nutrient pump compatible with the second category usually requires several sensors, with each sensor being able to detect one type of identification component. Therefore, such a nutrient pump can identify various types of infusion consumables, and the number of recognized types depends on the number of sensors.

There are, however, fewer infusion consumables in the first category, which limits the applicability of the nutrition pumps that are compatible with these consumables. Meanwhile, the second category of infusion consumables requires the corresponding nutrition pump to be equipped with multiple sensors, resulting in a more complex pump structure and higher costs.

### SUMMARY OF THE INVENTION

An object of this invention is to provide an infusion consumable, an infusion device and a method for detecting a model of the infusion consumable, which has a simple structure and is easily recognized, and which can solve the problem in the prior art that the structure of the infusion device that cooperates with the infusion consumable is complicated and costly, due to more sensors.

A first aspect of the invention provides an infusion consumable used in an infusion device, comprising a mounting portion, wherein the mounting portion is provided with a mounting hole, which is provided with an identification member, in which the depth of the mounting hole is greater than or equal to the thickness of an identification member.

In the infusion consumable in the embodiment of the invention, the identification member is provided to facilitate an infusion device used in conjunction with the infusion consumable to recognize the model of the infusion consumable, so as to enable the device to subsequently select an infusion mode corresponding to the infusion consumable. By providing a mounting hole on the mounting portion, a mounting position can be provided for the identification member. In addition, the manufacturing process needed to create the hole-like structure is relatively simple, which can reduce the processing cost of the infusion consumable. The depth of the mounting holes is greater than the thickness of the identification member, so that different models of the infusion consumable are differentiated according to the depth of the identification member mounted in the mounting holes. This has the effect of using a single identification member, which can identify a plurality of models of the infusion consumable. This can in turn simplify the structure of the infusion device corresponding to the infusion consumable, and thus reduce the cost of the infusion device.

In one embodiment, the identification member comprises a magnetic member.

In the infusion consumable in the embodiment of the invention, the identification member is provided with the magnetic member. The cost of the infusion consumable can thereby be reduced due to the lower cost of the magnetic member. In addition, the magnetic member has a polarity, so that different identification parameters can be provided for distinguishing different infusion consumables, which in turn provides the possibility of increasing the models of the infusion consumables. Furthermore, identification parameters may be provided for the identification of the infusion device, so that the infusion device recognizes different infusion consumables.

In one embodiment, the identification member is provided at the bottom of the mounting hole.

By providing the identification member at the bottom of the mounting hole, the distance from the top of the identification member to the opening of the mounting hole can be made different when the depth of the mounting hole is different. Thereby, identification parameters are provided for distinguishing between different models of infusion consumables, such as the height information of the identification member to the opening of the mounting hole or magnetic flux information of the identification member, in order for the infusion device to recognize infusion consumables based on different signals.

In one embodiment, a ratio of the depth of the mounting hole to the thickness (distance in the vertical dimension, viewed as in the figures) of the identification member is greater than or equal to 2.

By setting the ratio of the depth of the mounting holes to the thickness of the identification member to be greater than or equal to 2, it is possible to increase the number of mounting positions in the mounting holes of the identification member, which provides the possibility of increasing the number of distinguishable models of the infusion consumables when the models of the infusion consumables are distinguished by the difference in mounting positions.

In one embodiment, the mounting holes are provided with a limiting structure, which is configured to limit the identification member to different heights within the mounting hole.

In the infusion consumable in the embodiment of the invention, a limiting structure is provided in the mounting hole to limit the identification member to different heights in the mounting hole, which supports the identification member and prevents the identification member from shifting in the mounting hole, thereby ensuring that the infusion consumable can be used normally, and prolonging the service life of the infusion consumable.

In one embodiment, a plurality of the limiting structures are spaced apart along a depth direction of the mounting hole.

The infusion consumable in the embodiment of the invention is provided with a plurality of limiting structures and the plurality of limiting structures is provided at intervals along the depth direction of the mounting holes, so that the identification member can be provided at different heights in the mounting holes.

In one embodiment, the infusion consumable further comprises a fixing member, which is provided between the identification member and the mounting hole, and is configured to fixedly connect the identification member to the mounting hole.

In the infusion consumable in the embodiment of the invention, a fixing member is provided to enhance the solidity of the connection between the identification member and the mounting hole, and to extend the service life of the infusion consumable.

In one embodiment, the fixing member is a glue or an adhesive material piece.

In the infusion consumable in the embodiment of the invention, the fixing member, as a glue or an adhesive material piece, is provided to reduce the difficulty of installing the fixing member. In other words, the fixing member formed by the glue or the adhesive material can change with the shape of the identification member and the mounting hole, and then attach to the surface of the identification member and the mounting hole conveniently. In addition, the fixing member formed by the glue or the adhesive material may be a thinner layer, occupying a smaller volume, which in turn may reduce the influence of the fixing member on the identification member, and thus improve the identification accuracy of when cooperating with the infusion device.

In some embodiments, it is also possible to not provide a limiting structure or a fixing member. For example, the mounting hole may be provided as a tapered hole that becomes gradually larger from the bottom to the top, such that the identification member can be stably positioned where it “jams” in the mounting hole. In this way, the mounting height of the identification member in the mounting hole can be changed by changing the inner diameter of the mounting hole. In other embodiments, it is also possible to connect the identification member to the mounting hole in other ways.

In one embodiment, there is a plurality of the mounting holes, spaced apart on the mounting portion, with each of the mounting holes being configured to accommodate a respective identification member.

The infusion consumable in the embodiment of the invention is provided with a plurality of mounting holes and each mounting hole is configured to be provided with an identification member. This can further increase the number of distinct identification parameters for different infusion consumables, which in turn provides the possibility to increase the number of consumable models that the infusion device can identify. Additionally, more identification parameters may be provided for the identification of the infusion device, so that the infusion device recognizes different infusion consumables.

In one embodiment, the infusion consumable further comprises infusion tubing, which is fixedly connected to the mounting portion and is used to connect the infusion consumable to a nutrient pump.

The infusion consumable in an embodiment of the invention is provided with an infusion tubing so that the infusion consumable can be infused into a user.

A second aspect of the invention provides an infusion device comprising a consumable mounting slot and a consumable detection device. The consumable mounting slot is configured to mount any one of the above-described infusion consumables. The consumable detection device is provided opposite the identification member on the infusion consumable to identify a model of the infusion consumable.

In the infusion device in the embodiment of the invention, a consumable mounting slot is provided to mount the infusion consumable on the infusion device, in order to ensure that the infusion consumable can be stably connected to the infusion device when in use. The consumable detection device is provided to recognize the model of the infusion consumable mounted on the infusion device, so that the infusion device can determine an appropriate infusion mode according to the model of the infusion consumable.

In one embodiment, the identification member of the infusion consumable comprises a magnetic member. The consumable detection device in this case comprises:
a consumable detection member configured to detect a magnetic field strength and/or a magnetic direction of the identification member mounted in the infusion consumable; and
a controller configured to determine the model of the infusion consumable according to the magnetic field strength and/or the magnetic direction of the identification member.

In the infusion device in embodiments of the invention, the identification member is installed in a structure including a magnetic member, and the model of the predetermined infusion consumable is determined according to the magnetic field strength and/or magnetic direction of the identification member, so that the identification process is simplified because the method of detecting the magnetic field strength and/or magnetic direction is relatively simple. In addition, there are many types of devices that can recognize the magnetic field strength and/or magnetic direction, so the selection range of the consumable monitoring member can be expanded, which is conducive to the large-scale production of the infusion device, and also provides the possibility of reducing the cost of the infusion device.

A third aspect of this invention provides a method for identifying themodel of an infusion consumable, used in an infusion device, wherein the infusion device is provided with a consumable mounting slot configured to mount an infusion consumable. The infusion consumable in this case comprises a mounting portion, which is provided with a mounting hole, which is in turn provided with an identification member. The depth of the mounting hole is greater than or equal to the thickness of the identification member. The identification member comprising a magnetic member.

This method comprises:
detecting at least one of a magnetic field strength and a magnetic direction of the identification member mounted on the infusion consumable; and
determining a model of the infusion consumable according to at least one of the magnetic field strength and the magnetic direction of the identification member.

In the method for detecting a model of an infusion consumable in an embodiment of the invention, the identification member on the infusion consumable is installed in a structure that includes a magnetic member, and the model of the infusion consumable is determined according to the magnetic field strength and/or magnetic direction of the identification member. Since the way of detecting the magnetic field strength and magnetic direction is relatively simple, the difficulty of the identification can be reduced, and the process of identification is simplified. This improves identification efficiency and accuracy.

### BRIEF DESCRIPTION OF THE DRAWINGS

In order to more clearly illustrate the technical solutions in the embodiments, the accompanying drawings that need to be used in the description of the embodiments will be briefly introduced below. Obviously, the accompanying drawings in the following description are some of the embodiments of this invention, and for the person of ordinary skill in the field, other accompanying drawings may be obtained according to these drawings without paying creative labor.

FIG. 1 is a schematic structural diagram of an infusion consumable according to an embodiment of the invention.

FIG. 2 shows a schematic structural diagram of a mounting portion of the infusion consumable according to an embodiment of the invention.

FIG. 3 is a schematic structural diagram of an identification member of the infusion consumable according to an embodiment of the invention.

FIG. 4A is a sectional view of a mounting hole and an identification member of an infusion consumable according to an embodiment of the invention.

FIG. 4B is a sectional view of a mounting hole and an identification member of another infusion consumable according to an embodiment of the invention.

FIG. 4C is a sectional view of a mounting hole of an infusion consumable according to an embodiment of the invention.

FIG. 5 is a sectional view of a mounting hole of another infusion consumable according to an embodiment of the invention.

FIG. 6 is a sectional view of a mounting hole of yet another infusion consumable according to an embodiment of the invention.

FIG. 7 is a sectional view of a mounting hole of still another infusion consumable according to an embodiment of the invention.

FIG. 8 is a sectional view of a mounting hole and an identification member of an infusion consumable according to an embodiment of the invention.

FIG. 9 is a sectional view of a mounting hole and an identification member of another infusion consumable according to an embodiment of the invention.

FIG. 10 is a sectional view of a mounting hole and an identification member of yet another infusion consumable according to another embodiment of the invention.

FIG. 11 is a partial sectional view of a mounting portion of an infusion consumable according to an embodiment of the invention.

FIG. 12 is a schematic structural diagram of an infusion device according to an embodiment of the invention.

FIG. 13 is a schematic structural diagram of an infusion device with an infusion consumable mounted according to an embodiment of the invention.

FIG. 14 shows a schematic structural diagram of a consumable detection member of an infusion device according to an embodiment of the invention.

FIG. 15 is a flowchart of a method for detecting a model of an infusion consumable according to an embodiment of the invention.

FIG. 16 shows a flowchart of another method for detecting a model of an infusion consumable according to an embodiment of the invention.

### DESCRIPTION OF THE REFERENCE SIGNS

100: infusion consumable;
110: mounting portion;
111: mounting hole;
120: infusion tubing;
121: inlet end;
122: outlet end;
123: silicone hose portion;
130: identification member;
131: first end portion;
132: second end portion;
140: limiting structure;
150: fixing member;
200: infusion device;
210: consumable detection member;
220: controller;
230: consumable mounting slot;
240: consumable detection device.

### DETAILED DESCRIPTION

In order to make the objects, technical solutions and advantages of the embodiments of this invention clearer, the technical solutions in the embodiments of this invention will be described clearly and completely in the following in conjunction with the accompanying drawings in the embodiments of this invention. The description of embodiments below is not exhaustive but rather other embodiments obtained by a person of ordinary skill in the art without making creative labor fall within the scope of protection of this invention.

The terms “include” and “have” and any variations thereof in the specification and claims of this invention and the above-described drawings are intended to cover non-exclusive encompassing, for example, a process, method, system, product or apparatus including a series of steps or units need not be limited to those that are clearly listed, but may include other steps or units that are not clearly listed or are inherent to the process, method, product or apparatus.

In addition, in this invention, orientation terms such as “front,” “rear,” and the like are defined relative to the orientation in which the components are schematically placed in the accompanying drawings, and it should be understood that these orientation terms are relative concepts that are used for relative descriptions and clarifications, which may change accordingly to changes in the orientation in which the components are placed in the accompanying drawings.

In embodiments of the invention, “and/or”, which is merely an associative relationship describing an associated object, indicates that three kinds of relationships can exist, e.g., A and/or B, which can indicate the three cases of: the existence of A alone, the existence of both A and B, and the existence of B alone. In addition, the character “/” herein generally indicates that the associated objects are in an “or” relationship.

In the following, application scenarios of embodiments of this invention are described in conjunction with the accompanying drawings.

The invention provides an infusion consumable for use in an infusion device, which includes, but is not limited to, a nutrient pump and an infusion pump.

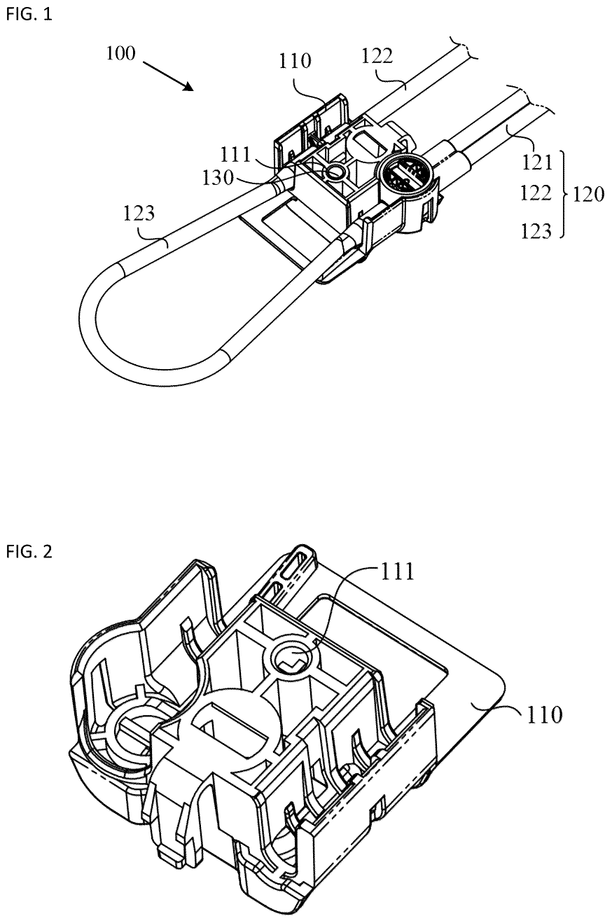

As shown in FIG. 1, the infusion consumable 100 may include a mounting portion 110 and infusion tubing 120, which is fixedly connected to the mounting portion 110, which in turn is configured to connect the infusion consumable 100 to the nutrient pump, so that the infusion consumable 100 can be infused into a user.

As an example, the infusion tubing 120 may include an inlet end 121 and an outlet end 122. A silicone hose portion 123 is provided between the inlet end 121 and the outlet end 122 and is connected to a roller of the infusion device. During use, the fluid may enter from the inlet end 121, and flow out from the outlet end 122 through the silicone hose portion 123. The outlet end 122 may be connected to a user.

It should be noted that the structure of the infusion tubing 120 includes, but is not limited to, the structure shown in the figure. The infusion tubing 120 may be different for different models of the infusion device. The infusion tubing 120 in embodiments of the invention is not further described.

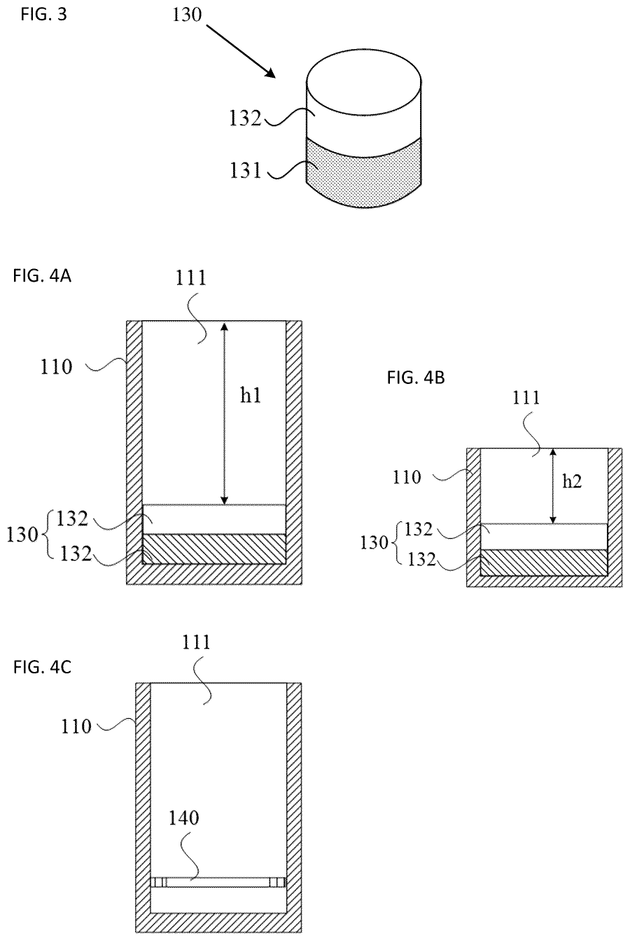

In some embodiments, as shown in FIG. 2, the mounting portion 110 is provided with a mounting hole 111, and an identification member 130 (FIG. 3) is provided in the mounting hole 111. A depth of the mounting hole 111 is greater than a thickness (the “height”, that is, length in the vertical direction, viewed as in FIG. 3) of the identification member 130. The identification members 130 on different models of the infusion consumable 100 are mounted at different depths within the mounting holes 111 and/or the identification members 130 on different models of the infusion consumable 100 are mounted in different orientations in the mounting holes 111.

As an example, the different mounting depths of the identification members 130 in the mounting holes 111 on the different models of the infusion consumable 100 refer to the feature that different identification members 130 have different heights from the top of the member to the opening of the mounting hole 111, or different identification members 130 have different heights from the bottom end of the member to the bottom end of the mounting holes 111.

As an example, the identification member 130 may include a first end portion 131 and a second end portion 132. In this case, the different mounting orientations of the identification members 130 in the mounting holes 111 on different models of the infusion consumable 100 refer to the feature that the orientations of the first end portions 131 of the identification members 130 on different models of the infusion consumable 100 are different. For example, the first end portion 131 may be oriented toward a bottom end of the mounting hole 111 in one direction and the first end portion 131 is oriented toward an open end of the mounting hole 111 in another direction.

In the infusion consumable 100 in the embodiment of the invention, the identification member 130 is provided to facilitate the identification of a model of the infusion consumable 100 by an infusion device used in conjunction with the infusion consumable 100, so as to subsequently select an infusion mode corresponding to the infusion consumable 100. By providing a mounting hole 111 on the mounting portion 110, a mounting location can be provided for the identification member 130. In addition, the manufacturing process of the hole structure is relatively simple, which can reduce the processing cost of the infusion consumable 100.

By using the depth of the identification member 130 provided in the mounting holes 111 to distinguish different models of the infusion consumable 100, the effect of using one identification member 130 to identify multiple models of the infusion consumable 100 can be realized. Furthermore, the structure of the infusion device corresponding to the infusion consumable 100 can be simplified, thereby reducing the cost of the infusion device.

In addition, by setting the depth of the mounting holes 111 greater than the thickness of the identification member 130, the identification member 130 can be invisibly mounted in the infusion consumable 100, thereby preventing the problem of the identification member 130 mistakenly being touched during use and damaging the identification member 130.

In some embodiments, as shown in FIG. 3, the identification member 130 may be formed as a cylindrical structure. In other embodiments, the identification member 130 may be formed as a structure that has other shapes, such as a three-edged column, a four-edged column, etc. Embodiments of the invention are not limited to any specific shape for the identification member 130.

By forming the identification member 130 as a cylindrical structure, the manufacturing procedure of the identification member 130 can be simplified, thereby reducing the production cost of the infusion consumable 100.

As shown in FIG. 3, the identification member 130 includes two end portions, respectively a first end portion 131 and a second end portion 132. The first end portion 131 and the second end portion 132 have different features.

By making the features of the two end portions of the identification member 130 different, when the identification member 130 is mounted, the mounting orientation can be distinguished, and thus mounting errors can be prevented. Furthermore, the indication of the model or type of the infusion device can be changed by changing the mounting orientation of the identification member 130.

It will be appreciated that the two ends of the identification member 130 are oriented differently in the mounting holes 111, which indicates that the mounting orientation of the identification member 130 in the mounting holes 111 may different. For example, the first end portion 131 of the identification member 130 may be oriented toward the bottom end of the mounting hole 111 so that the mounting orientation is opposite to the mounting orientation of the second end portion 132 of the identification member 130 toward the bottom end of the mounting hole 111.

In some embodiments, the identification member 130 may include or be a magnetic member. The magnetic member will then include two poles, a south pole and a north pole, located at the opposite ends of the magnetic member. The two ends of the identification member 130 including the magnetic member are characterized differently due to the different orientations of the magnetic field generated by the south and north poles.

In the infusion consumable 100 in the embodiment of the invention, the identification member 130 is provided as a structure including a magnetic member, which can reduce the cost of the infusion consumable 100 due to the relatively low cost of the magnetic member. In addition, the magnetic member, being magnetic, has a polarity, so it can provide different identification parameters for distinguishing different infusion consumables 100, which in turn provides the possibility of increasing the number of models of the infusion consumables 100. In addition, identification parameters may be provided for the identification of the infusion device, so that the infusion device recognizes the different infusion consumables 100.

In some embodiments, the identification member 130 may also be formed as a cylindrical structure with a magnetic member provided at one end. In other embodiments, the identification member 130 may also be formed a structure of other materials. Embodiments of the invention are not limited to any specific structure of the identification member 130.

In some embodiments, a ratio of a depth of the mounting holes 111 to a thickness of the identification member 130 may be greater than or equal to 2. As an example, the depth of the mounting holes 111 may be 2 times, 2.5 times, 3 times, 3.5 times, 4 times, or more times the thickness of the identification member 130.

Embodiments of the invention are not limited to any particular quantitative relationship between the ratio of the depth of the mounting hole 111 and the thickness of the identification member 130.

It is to be understood that the proportional relationship between the thickness of the identification member 130 and the depth of the mounting hole 111 may determine the number of models of the infusion consumable 100. That is, by changing the ratio of the depth of the mounting hole 111 to the thickness of the identification member 130, the number of mounting positions in the mounting holes 111 of the identification member 130 can be changed, and thus the number of models of the infusion consumable can be changed. The ratio of the depth of the mounting hole 111 to the thickness of the identification member 130 may be set according to specific needs and actual circumstances.

As shown in FIG. 4A, the mounting hole 111 may be formed as a hole-like structure with a cylindrical shape.

It is to be noted that the cylindrical hole-like structure is easy to manufacture, which can simplify the processing of the mounting hole 111 and thereby reduce the production cost of the infusion consumable 100.

In some other embodiments, the mounting hole 111 may also be formed as structures of other shapes, such as, a triangular-prismatic, or a quadrangular-shaped aperture structure, which may be chosen according to the specific structure of the identification member 130. Embodiments of the invention are not limited to any specific structure of the mounting holes 111.

As shown in FIG. 4A, the identification member 130 is disposed at the bottom of the mounting hole 111. The depths of the mounting holes 111 varies from model to model of the infusion consumable 100.

As an example, as shown in FIGS. 4A and 4B, both models of the infusion consumable 100 have the same thickness of the identification member 130 and different depths of the mounting holes 111. The identification member 130 of the infusion consumable 100 of both models is provided at the bottom of the mounting hole 111 such that the distance h1 from the second end portion 132 of the identification member 130 of the infusion consumable 100 to the opening of the mounting hole 111 in FIG. 4A is different from the distance h2 from the second end portion 132 of the identification member 130 of the infusion consumable 100 to the opening of the mounting hole 111 in FIG. 4B.

In the embodiments of the invention, there is no limitation as to the depth of the mounting hole 111 and the size of the identification member 130, for example, the depth of the mounting hole 111 may be 2 mm, 3 mm, 4 mm, 5 mm, 6 mm, etc., and the thickness of the identification member 130 may be 1 mm, 2 mm, 3 mm, etc.

In this way, by mounting the identification member 130 at the bottom of the mounting holes 111 and setting the depth of the mounting holes 111 differently for different models of the infusion consumables 100, it is possible to make the distance from the top of the identification member 130 to the opening of the mounting holes 111 different for different models of the infusion consumables 100. This can provide identification parameters, such as, height information or magnetic flux information, for the identification of the infusion device so that the infusion device recognizes different infusion consumables.

In some other embodiments, it is also possible to set the depths of the mounting holes 111 of the different models of the infusion consumables 100 to be the same, and to set the thicknesses of the identification members 130 to be the same, so that the identification members 130 are fixed to different depths of the mounting holes 111 by changing the structure of the mounting holes 111.

As an example, as shown in FIG. 4C, the mounting holes 111 may be provided with a limiting structure 140, which is configured to limit the identification member 130 to different heights within the mounting hole 111.

In the infusion consumable 100 in the embodiment of the invention, the limiting structure 140 is provided in the mounting holes 111 to limit the identification member 130 to different heights in the mounting holes 111, which support the identification member 130 and prevent it from shifting in the mounting holes 111, thereby ensuring that the infusion consumable 100 can be used normally, and prolonging the service life of the infusion consumable 100.

As an example, the limiting structure 140 may be an annular plate-like structure provided in the mounting holes 111, wherein an outer edge of the annular plate-like structure is connected to an inner wall of the mounting holes 111, and an inner edge of the annular plate-like structure extends in a direction to a center axis of the mounting holes 111. The inner edge of the annular plate-like structure has a diameter that is larger than an outer diameter of the identification member 130, so that the limiting structure 140 may provide support for the identification member 130. In some other embodiments, the limiting structure 140 may also be a plate-like structure provided within the mounting hole 111, and an outer wall of the plate-like structure is fixedly connected to an inner wall of the mounting hole 111.

In some other embodiments, as shown in FIG. 5, the limiting structure 140 may also be provided as a screw structure. As an example, the limiting structure 140 is arranged through the side wall of the mounting hole 111 in a vertical direction along a center axis of the mounting hole 111 and is movably connected to the mounting hole 111. The length of the limiting structure 140 is greater than the wall thickness of the mounting holes 111. Rotating the limiting structure 140 in different directions can move the limiting structure 140 in a direction close to or away from the inside of the mounting hole 111.

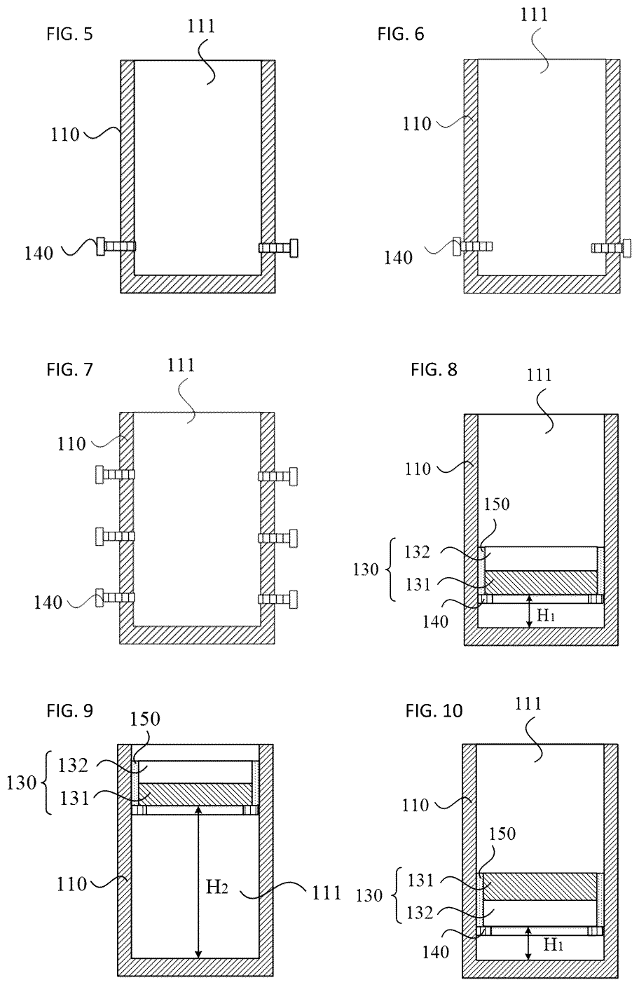

As shown in FIG. 6, the limiting structure 140 is a screw, and the limiting structure 140 is rotated until a portion of the structure extends into the mounting hole 111. As shown in FIG. 6, when the limiting structure 140 is rotated until a portion of the structure is located within the mounting hole 111, it can act as a limiter for the identification member 130.

Embodiments of the invention are not limited to any particular number of limiting structures 140 disposed at the same height; for example, the limiting structures 140 disposed at the same height may be a plurality of limiting structures, and the plurality of limiting structures 140 may be spaced apart around the mounting hole 111.

Alternatively, the limiting structure 140 disposed at the same height may be one, which is disposed on the outside of the mounting hole 111. The limiting structure 140 may have a larger size, e.g., the size of the limiting structure 140 may be larger than the radius of the mounting hole 111.

Embodiments of the invention are not limited to any specific structure of the limiting structure 140, or number of limiting structures 140 at the same height. As long as the limiting structure 140 can realize this limiting function, it falls within the scope of protection of the embodiments of the invention.

In one embodiment, as shown in FIG. 7, the plurality of limiting structures 140 is spaced apart along the depth direction of the mounting hole 111.

The infusion consumable 100 in the embodiment of the invention is provided with a plurality of limiting structures 140, which are provided at intervals along the depth direction of the mounting hole 111, so as to realize that the identification member 130 is provided at different heights in the mounting hole 111.

In one embodiment, as shown in FIG. 8, the infusion consumable 100 may further include a fixing member 150. The fixing member 150 is provided between the identification member 130 and the mounting holes 111, and the fixing member 150 is configured to fixedly connect the identification member 130 to the mounting holes 111. As an example, the fixing member 150 may be glue or an adhesive material piece.

In the infusion consumable 100 in the embodiment of the invention, the fixing member 150 is provided to enhance the solidity of the connection between the identification member 130 and the mounting holes 111, thereby extending the service life of the infusion consumable 100.

In the infusion consumable 100 in the embodiment of the invention, by choosing the fixing member 150 to be a glue or an adhesive material piece, the difficulty of setting the fixing member 150 can be reduced. In other words, the fixing member 150 formed by the glue or the adhesive material piece can change with the shape of the identification member 130 and the mounting holes 111 and attach to the surfaces of the identification member 130 and the mounting holes 111 conveniently. In addition, the fixing member 150 formed by the glue or the adhesive material may be a thinner layer, occupying a smaller volume, which in turn may reduce the influence of the fixing member 150 on the identification member 130, and thus improve the accuracy of being recognized when cooperating with the infusion device.

In other embodiments, the fixing member 150 may also be in other forms, for example, it may be a rubber ring and the like. Embodiments of the invention are not limited to any specific structure of the fixing member 150.

It is to be noted that the infusion consumable 100 in the embodiments of the invention may be distinguished and its model identified by the different depths at which the identification member 130 is provided in the mounting holes 111 and/or the mounting orientation of the identification member 130.

That is to say, the height from the bottom end of the identification member 130 to the bottom end of the mounting hole 111 is different for different models of the infusion consumable 100 and/or the mounting orientation of the identification member 130 in the mounting hole 111 is different for different models of the infusion consumable 100.

In other words, different models of the infusion consumables 100 may be distinguished by the depth of the identification member 130 provided in the mounting holes 111; alternatively, different models of the infusion consumables 100 may be distinguished by the mounting orientation of the identification member 130 in the mounting holes 111. As still another option, different models of the infusion consumables 100 may be distinguished by the depth of the identification member 130 provided in the mounting hole 111 and the mounting orientation of the identification member 130 in the mounting hole 111.

As an example, as shown in FIG. 8, a height from the bottom end of the identification member 130 to a bottom end of the mounting hole 111 is a first height H1, and a first end portion 131 of the identification member 130 is oriented toward the bottom end of the mounting hole 111.

As shown in FIG. 9, the vertical distance (height) from the bottom end of the identification member 130 to the bottom end of the mounting hole 111 is a second height H2, and the first end portion 131 of the identification member 130 is oriented toward the bottom end of the mounting holes 111.

FIGS. 8 and 9 illustrate the mounting holes 111 and the identification members 130 provided on two models of infusion consumables 100, in which the identification members 130 on both infusion consumables 100 are mounted in the same orientation and at different heights in the mounting holes 111.

As shown in FIG. 10, the vertical distance from the bottom end of the identification member 130 to the bottom end of the mounting hole 111 is the first height H1, and the second end 132 of the identification member 130 is oriented toward the bottom end of the mounting hole 111.

FIGS. 8 and 10 illustrate the mounting holes 111 and the identification members 130 provided on two models of infusion consumables 100, in which the identification members 130 on both infusion consumables 100 are mounted in different orientations and at the same height in the mounting holes 111.

FIGS. 9 and 10 illustrate the mounting holes 111 and the identification members 130 provided on two models of infusion consumables 100, in which the identification members 130 on both infusion consumables 100 are mounted in different orientations and at different heights in the mounting holes 111.

In this way, it is possible to mark different models of the infusion consumable 100 by changing the mounting position and orientation of the identification member 130 on the infusion consumable 100, thus offering the possibility of increasing the number of separately identifiable models of the infusion consumable 100, and thus increasing the range of applicability of the infusion consumable 100.

In one embodiment, as shown in FIGS. 8-10, a single-channel infusion consumable and a dual-channel infusion consumable may be distinguished by the mounting orientation. Different models of infusion consumables for the same channel are distinguished by the mounting depth. The dual-channel infusion consumable is provided with one more inlet tube relative to the single-channel infusion consumable, which is located at the inlet end of the infusion consumable.

As an example, the infusion consumable with the first end portion 131 of the identification member 130 facing the bottom end of the mounting hole 111 may be regarded as the single-channel infusion consumable. When the first end portion 131 of the identification member 130 faces the bottom end of the mounting hole 111 and the depth of the identification member 130 in the mounting hole 111 is different, it may indicate that the two single-channel infusion consumables are of different models.

The infusion consumable with the second end portion 132 of the identification member 130 facing the bottom end of the mounting hole 111 may be regarded as a dual-channel infusion consumable. When the second end portion 132 of the identification member 130 is oriented toward the bottom end of the mounting hole 111 and the depth of the identification member 130 in the mounting hole 111 is different, it may indicate that the two dual-channel infusion consumables are of different models.

It should be noted that the different depths of the identification member 130 provided in the mounting holes 111 can be realized by changing the depth of the mounting holes 111, or by changing the mounting position of the identification member 130 within the mounting holes 111. Embodiments of the invention are not limited to any specific means by which the depth of the identification member 130 within the mounting holes 111 is changed.

In one embodiment, as shown in FIG. 11, there is a plurality of mounting holes 111. The plurality of mounting holes 111 is spaced apart on the mounting portion 110, and each mounting hole 111 is configured to accommodate an identification member 130.

In the infusion consumable 100 in the embodiment of the invention, a plurality of mounting holes 111 is provided, and each mounting hole 111 is configured to accommodate an identification member 130. This can further increase the number of different identification parameters for distinguishing different infusion consumables 100, and thus provide the possibility of increasing the number of separately identifiable models of the infusion consumable 100. In addition, more identification parameters can be provided for the identification of the infusion device, so that the infusion device recognizes different infusion consumables 100.

It is to be noted that in the embodiments of the invention, the number of mounting holes 111 may be 1, 2, 3, 4, 5 or more, which may be set according to the practical demand.

In addition, the plurality of mounting holes 111 may be arranged in an array on one side of the mounting portion 110, or may be irregularly arranged. Embodiments of the invention are not limited to any specific arrangement for the plurality of mounting holes 111.

It is to be noted that different models of the infusion consumable 100 (e.g., a dual-channel infusion consumable 100 and a single-channel consumable) may be adapted to different application scenarios, thus expanding the range of detectable models of the infusion consumable 100. Furthermore, the mounting orientation and the mounting height of the identification member 130 may be used as a reference for differentiation, thereby reducing the difficulty of differentiating the models of the consumables and the cost. Moreover, the cost of the infusion device corresponding to the infusion consumable 100 can also be reduced.

The infusion device 200 according to the embodiments of the invention is described below in conjunction with the accompanying drawings.

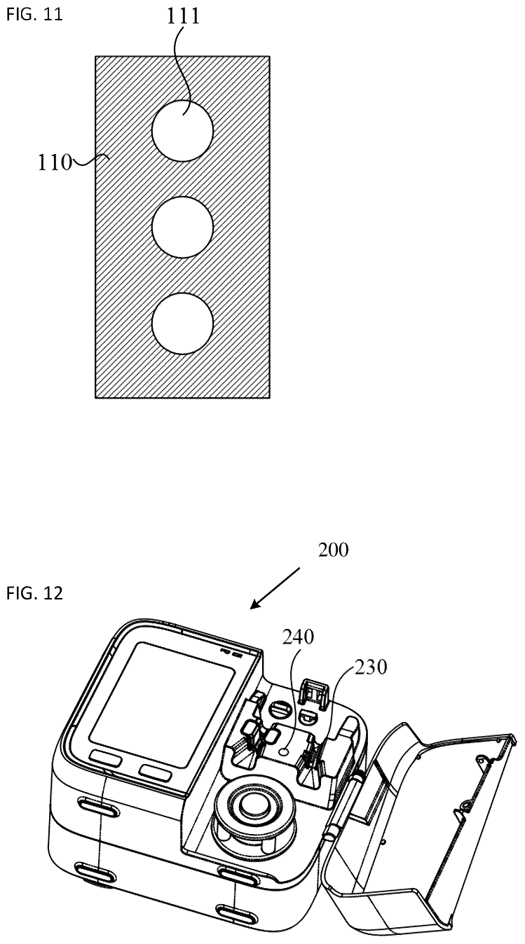

As shown in FIG. 12, the invention provides an infusion device 200, which may include a consumable mounting slot 230 and a consumable detection device 240. As shown in FIG. 13, the consumable mounting slot 230 is configured to mount the above-described infusion consumable 100. The consumable detection device 240 is provided relative to the identification member 130 of the infusion consumable 100 for identifying a model of the infusion consumable 100.

The infusion device 200 in the embodiment of the invention is provided with the consumable mounting slot 230 so as to mount the infusion consumable 100 to the infusion device 200, thereby ensuring that the infusion consumable 100 can be stably connected to the infusion device 200 during use. The consumable detection device 240 is provided so as to recognize the model of the infusion consumable 100 mounted on the infusion device 200, so that the infusion device 200 can determine a suitable infusion mode according to the model of the infusion consumable 100.

In some embodiments, the identification member 130 of the infusion consumable 100 is a magnetic member.

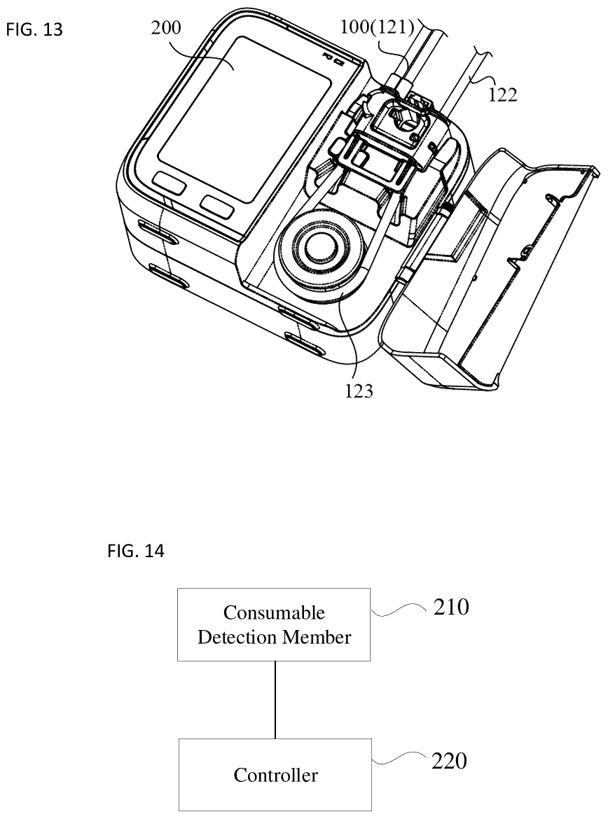

As shown in FIG. 14, the consumable detection device 240 may include a consumable detection member 210 and a controller 220.

The consumable detection member 210 is configured to detect a magnetic field strength and/or a magnetic direction of the identification member 130 on the infusion consumable 100.

The controller 220 is configured to determine a model of the infusion consumable 100 according to the magnetic field strength and/or the magnetic direction of the identification member 130.

As an example, the consumable detection member 210 may be electrically connected to the controller 220, so that the controller 220 can determine the model of the infusion consumable 100 according to the magnetic field strength and/or the magnetic direction of the identification member 130.

In the infusion device 200 in some embodiments of the invention, the identification member 130 may be a magnetic member, and the model of the infusion consumable 100 may be determined according to the magnetic field strength and/or magnetic direction (orientation relative to polarity) of the identification member 130. This can simplify the identification process because the way of detecting the magnetic field strength and/or magnetic direction is relatively simple. In addition, there are many types of devices that can recognize the magnetic field strength and/or magnetic direction, so the selection range of the consumable monitoring member can be expanded, which is conducive to the large-scale production of the infusion device 200, and also provides the possibility of reducing the cost of the infusion device 200.

In some embodiments, the controller 220 is further configured to:
store model information of a plurality of models of the infusion consumables 100;
compare the magnetic field strength and/or the magnetic direction information detected by the consumable detection member 210 with the model information; and
determine the model of the infusion consumable 100 according to the comparison results.

In some embodiments, the controller 220 is further configured such that if the magnetic field strength and/or the magnetic direction information detected by the consumable detection member 210 is the same as one of the model information, the model corresponding to the model information is determined as the model of the infusion consumable.

It is to be noted that the model information may be the magnetic field strength and/or magnetic direction information corresponding to the infusion consumable 100. One model of the infusion consumable 100 corresponds to one model information, and different models of the infusion consumable 100 correspond to different model information.

In some embodiments, the consumable detection member 210 may be a sensor, e.g., a magnetic inductive sensor, a linear Hall sensor or a magnetoresistive sensor. Embodiments of the invention are not restricted to any specific structure of the consumable detection member 210.

In some embodiments, the consumable detection member 210 may also be configured to detect the mounting orientation and mounting depth of the identification member 130 of the infusion consumable 100 in the mounting hole 111. The controller 220 is configured to determine a model of the infusion consumable 100 according to the mounting orientation and mounting depth of the identification member 130. As an example, the consumable detection member 210 may be a distance sensor or the like. Embodiments of the invention are not limited with respect to the information acquired by the consumable detection member 210.

It is to be noted that the infusion device 200 is also provided with other structures such as a housing, or a display screen. Embodiments of the invention do not presuppose or require any particular type of form for such structures.

Embodiments of the invention also provide a method for detecting a model of an infusion consumable that is used in an infusion device, which is provided with a consumable mounting slot configured to mount an infusion consumable. The infusion consumable includes a mounting portion, which is provided with a mounting hole. An identification member is provided in the mounting hole, and a depth of the mounting hole is greater than a thickness of the identification member. The identification member is a magnetic member.

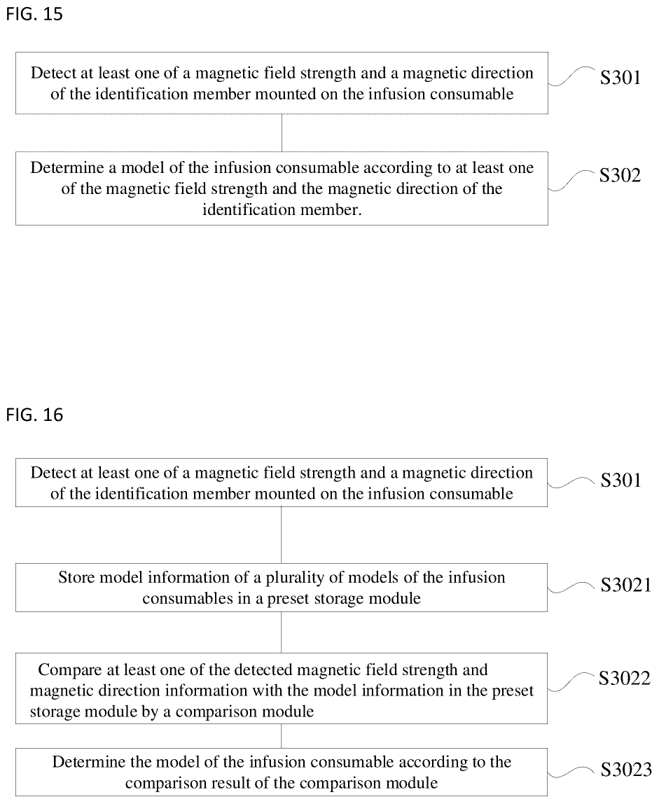

FIG. 15 shows a flowchart of a method for detecting a model of an infusion consumable according to an embodiment of the invention.

The method is carried out by a processor included in the infusion device. The method for detecting the model of the infusion consumable is described below with the infusion device as the apparatus that carries out the various method steps.

As shown in FIG. 15, the method includes the following steps.

S301: a magnetic field strength and/or a magnetic direction of the identification member on the infusion consumable is detected.

A detailed description is provided below for S301 is as follows.

Since there are a variety of models of infusion consumables, and the identification members of different models of the infusion consumables are mounted in different directions and/or in different positions, the magnetic field strength and direction can be varied. Since the magnetic field strength and magnetic direction can be detected and evaluated relatively easily, this can reduce the difficulty of identification, simplify the identification process, enhance the efficiency of identification, and improve the accuracy of identification.

In some embodiments, before detecting the magnetic field strength and/or magnetic direction of the identification member on the infusion consumable, the method further includes: mounting the infusion consumable to a consumable mounting slot of the infusion device. The consumable mounting slot is configured for fixing in place the infusion consumable, so that the accuracy of the detection results can be improved.

S302: the model of the infusion consumable is determined according to the magnetic field strength and/or the magnetic direction of the identification member.

A detailed description is provided below for S302 is as follows.

As shown in FIG. 16, in some embodiments, the step of determining the model of the infusion consumable according to the magnetic field strength and/or magnetic direction of the identification member specifically includes the following steps.

S3021: model information of a plurality of models of the infusion consumables is stored as respective data entries in a preset storage module.

S3022: the detected magnetic field strength and/or magnetic direction information is compared with the model information in the preset storage module by a comparison module.

S3023: the model of the infusion consumable is determined according to the comparison result of the comparison module.

In some embodiments, the step of determining the model of the infusion consumable according to the comparison result of the comparison module includes: if the detected magnetic field strength and/or magnetic direction information is the same as one of the model information entries, the model corresponding to the model information is determined as the model of the infusion consumable.

It should be noted that in some embodiments in which the identification member does not include a magnetic member, the method can still be used to determine the model of the infusion consumable. For example, the mounting orientation and/or the mounting depth of the identification member on the infusion consumable in the mounting hole can be directly detected; then the model of the infusion consumable can be determined according to the mounting orientation and/or the mounting depth of the identification member. It is sufficient that different models of the infusion consumables can be distinguished by the mounting depth and the mounting orientation.

In the method for detecting the model of the infusion consumable in the embodiment of the invention, the identification member of the infusion consumable is attached to a structure including a magnetic member, and the model of the infusion consumable is determined according to the magnetic field strength and/or magnetic direction of the identification member. Since the way of detecting and judging the magnetic field strength and direction is relatively simple, this can reduce the difficulty of identification, simplify the identification process, enhance the efficiency of the identification, and improve the accuracy of the identification.

In the description of the embodiments of the invention, it is to be noted that, unless otherwise expressly provided and limited, the terms “mount”, “connect”, and “set” are to be broadly construed, for example, it may be a fixed connection, an indirect connection through an intermediate medium, a connection within two elements or an interactive relationship between two elements. For a person of ordinary skill in the art, the specific meaning of the above terms in the embodiments of the invention may be understood according to the specific circumstances.

The terms “first”, “second”, “third”, “fourth” and the like (if present) in the specification and the claims of the embodiments of the invention and the above-described accompanying drawings may be understood in the specific meaning in the embodiments of the invention.

Finally, it should be stated that the above embodiments are only used to illustrate the technical solution of this invention, not to limit it. Although this invention is described in detail with reference to the foregoing embodiments, a person of ordinary skill in the art should understand that: he or she can still make modifications to the technical solution documented in the foregoing embodiments, or carry out equivalent substitutions of some or all of the technical features therein. These modifications or substitutions that does not take the essence of the corresponding technical solutions out of the scope of the technical solutions of the embodiments of this invention.

The invention claimed is:

1. An infusion consumable for use in an infusion device, comprising a mounting portion, wherein the mounting portion is provided with a mounting hole, which is provided with an identification member; and
a depth of the mounting hole is greater than or equal to a thickness of the identification member; and
a detection device configured to identify the infusion consumable mounted on the infusion device by sensing a mounting characteristic of the identification member relative to the mounting hole.

2. The infusion consumable of claim 1, wherein the identification member comprises a magnetic member, in which the mounting characteristic is a magnetic property of the magnetic member.

3. The infusion consumable of claim 2, in which the magnetic property is polarity of the magnetic member.

4. The infusion consumable of claim 2, in which the magnetic property is magnetic field strength of the magnetic member.

5. The infusion consumable of claim 1, in which the mounting characteristic is a mounting depth of the identification member within the mounting hole.

6. The infusion consumable of claim 1, wherein the identification member is provided at the bottom of the mounting hole.

7. The infusion consumable of claim 1, wherein a ratio of the depth of the mounting hole to the thickness of the identification member is greater than or equal to 2.

8. The infusion consumable of claim 7, wherein the mounting hole is provided with a limiting structure, which is configured to limit the identification member to different depths within the mounting hole.

9. The infusion consumable of claim 8, wherein a plurality of the limiting structures are spaced apart along a depth direction of the mounting hole.

10. The infusion consumable of claim 1, further comprising a fixing member;
wherein the fixing member is provided between the identification member and the mounting hole, and the fixing member is configured to fixedly connect the identification member to the mounting hole.

11. The infusion consumable of claim 10, wherein the fixing member is glue or an adhesive material piece.

12. The infusion consumable of claim 1, wherein:
there is a plurality of the mounting holes;
wherein the plurality of the mounting holes is spaced apart on the mounting portion; and
each of the mounting holes is configured to accommodate one identification member.

13. The infusion consumable of claim 12, further comprising infusion tubing that is fixedly connected to the mounting portion, and the mounting portion is configured to connect the infusion consumable to a nutrient pump.

14. An infusion device, comprising:
a consumable mounting slot configured to mount the infusion consumable, the infusion consumable comprising:
a mounting portion, wherein the mounting portion is provided with a mounting hole, which is provided with an identification member; and a depth of the mounting hole is greater than or equal to a thickness of the identification member; and
a consumable detection device, which is provided opposite the identification member on the infusion consumable to identify a model of the infusion consumable.

15. The infusion device of claim 14, wherein the identification member of the infusion consumable comprises a magnetic member;
the consumable detection device comprises:
a consumable detection member configured to detect at least one of a magnetic field strength and a magnetic direction of the identification member mounted in the infusion consumable; and
a controller configured to determine the model of the infusion consumable according to the at least one of the magnetic field strength and the magnetic direction of the identification member.

16. A method for detecting a model of an infusion consumable used in an infusion device, wherein the infusion device is provided with a consumable mounting slot configured to mount an infusion consumable;
the infusion consumable comprises a mounting portion, wherein the mounting portion is provided with a mounting hole, which is provided with an identification member; a depth of the mounting hole is greater than or equal to a thickness of the identification member; and the identification member comprising a magnetic member;
the method comprises:
detecting at least one of a magnetic field strength and a magnetic direction of the identification member mounted on the infusion consumable; and
determining a model of the infusion consumable according to at least one of the magnetic field strength and the magnetic direction of the identification member.
